(19) United States
(12) Patent Application Publication
(10) Pub. No.: US 2022/0125960 A1
(43) Pub. Date: Apr. 28, 2022

(54) COMBINATION OF PD-1/PD-L1 INHIBITORS AND TARGETED THORIUM CONJUGATES

(71) Applicants: Bayer Aktiengesellschaft, Leverkusen (DE); Bayer AS, Oslo (NO)

(72) Inventors: Urs Beat HAGEMANN, Glienicke/Nordbahn (DE); Pascale LEJEUNE, Toulouse (FR); Jenny KARLSSON, Oslo (NO); Alan CUTHBERTSON, Oslo (NO); Stefanie HAMMER, Berlin (DE)

(73) Assignees: Bayer Aktiengesellschaft, Leverkusen (DE); Bayer AS, Oslo (NO)

(21) Appl. No.: 17/310,758

(22) PCT Filed: Feb. 17, 2020
(86) PCT No.: PCT/EP2020/054112

§ 371 Date: Aug. 20, 2021

(30) Foreign Application Priority Data
Feb. 21, 2019 (EP) 19158547.0

(51) Int. Cl. A61K 51/10; A61K 45/06; C07K 16/28; A61P 35/00

(52) U.S. Cl. CPC A61K 51/1072; A61K 45/06; A61K 51/1027; A61P 35/00; C07K 16/2818; C07K 16/2827

(57) ABSTRACT

The present invention relates to combinations of at least two components, component A and component B, component A being a PD-1/PD-L1 inhibitor, and component B being a targeted thorium conjugate. Another aspect of the present invention relates to the use of such combinations as described herein for the preparation of a medicament for the treatment or prophylaxis of a disease, particularly for the treatment of breast and prostate cancer.

The present invention relates to combinations of at least two components, component A and component B, component A being a PD-1/PD-L1 inhibitor, and component B being a targeted thorium conjugate.

Another aspect of the present invention relates to the use of such combinations as described herein for the preparation of a medicament for the treatment or prophylaxis of a disease, particularly for the treatment of cancer.

Yet another aspect of the present invention relates to methods of treatment or prophylaxis of a cancer in a subject, comprising administering to said subject a therapeutically effective amount of a combination as described herein.

Further, the present invention relates to a kit comprising a combination of:
one or more components A, as defined herein, or a physiologically acceptable salt, solvate, hydrate or stereoisomer thereof;
a component B, as defined supra, or a solvate or hydrate thereof; and, optionally
one or more pharmaceutical agents C;
   in which optionally either or both of said components A and B are in the form of a pharmaceutical formulation which is ready for use to be administered simultaneously, concurrently, separately or sequentially.

Component A may be administered by the oral, intravenous, topical, local installations, intraperitoneal or nasal route.

Component B preferably is administered by the intravenous route.

### BACKGROUND TO THE INVENTION

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for treating diseases associated with dysregulated growth/proliferation.

Cancer is a complex disease arising after a selection process for cells with acquired functional capabilities like enhanced survival/resistance towards apoptosis and a limitless proliferative potential. Thus, it is preferred to develop drugs for cancer therapy addressing distinct features of established tumors.

Recently, the PD-1/PD-L1 signalling pathway has emerged as important regulator of the activity of the immune system. In cancer, tumor cells express PD-L1, the ligand of PD-1, by which they can evade their killing by the host immune system. Inhibitors against PD-1 and its ligands PD-L1 and PD-L2 have recently been developed which interfere with this immune-suppressive mechanism and have shown amazing clinical efficacy, by extension of the overall survival of patients with various types of cancer. Some of these inhibitors have been approved for various cancer indications such as melanoma, NSCLC, HNSCC, RCC, bladder cancer and NHL. A large number of additional clinical trials are in progress in other indications and/or in combination with a variety of other antitumor agents in order to improve the therapeutic activity (Iwai et al, J. Biomedical Sci. (2017) 24:26, 1-11; Sweis and Luke, Pharm. Res. (2017) 120, 1-9; Bersanelle and Buti, World Journal of Clinical Oncology, (2017) 8(1), 37-53; Park et al., Arch. Pharm. Res. (2016) 39, 1577-1587).

PD-1 inhibitors are biologics, primarily immunoglobulins of the G subclass, which bind to programmed cell death protein 1 also known as PD-1 and block its activity. Known PD-1 inhibitors are nivolumab (Opdivo, BMS-936558, MDX1106), pembrolizumab (Keytruda, MK-3475, lambrolizumab), PDR-001, JS001 and STI-A1110.

PD-1 (also known as CD279) is a receptor protein which is expressed as monomer on the surface of various immune cells mainly on activated CD4+ and CD8+ T cells, on macrophages and on activated B cells, but was also found on natural killer (NK) cells and antigen presenting cells (APC). The extracellular domain of this type I membrane protein consists of a single IgV-like domain, followed by a transmembrane domain and a cytoplasmic region, which contains an immunoreceptor tyrosine-based inhibitory and switch motifs (ITIM and ITSM). Upon binding to its ligand PD-L1 or PD-L2, the phosphatase SHP-2 is recruited which dephosphorylates the kinase ZAP70, a major component of the T cell receptor (TCR) signaling complex. This shuts down TCR signaling and inhibits the cytotoxic activity of the T cells, their interferon gamma production and proliferation. In addition, PD-1 ligation up-regulates E3-ubiquitin ligases CBL-b and c-CBL that trigger T cell receptor down-modulation. PD-1 is encoded by the Pdcd1 gene in humans and is transcriptionally activated by transcription factors NFATc1, IRF9 and Fox01, which are activated upon TCR activation and by T cell exhaustion signals such as transforming growth factor ß and eomesodermin. The activation induced expression of PD-1 suggests that this receptor regulates rather the later phase of the immune response in the peripheral tissue (effector phase, memory response and chronic infection). This is in contrast to CTLA-4, another immune check point protein, which is more active in the earlier priming phase of the immune response and inhibitors of CTLA-4 (e.g. ipilimumab) appear to be less well tolerated in patients. (Iwai et al, J. Biomedical Sci. (2017) 24:26, 1-11; Sweis and Luke, Pharm. Res. (2017) 120, 1-9; Park et al., Arch. Pharm. Res. (2016) 39, 1577-1587).

PD-L1 inhibitors are biologics, primarily immunoglobulins of the G subclass, which bind to the ligand of PD-1 and block its activity. Known PD-L1 inhibitors are atezolizumab (Tecentriq, MPDL3280A), durvalumab (MED14736), avelumab (MSB0010718C), BMS-936559 (MDX1105) and LY3300054.

PD-L1 (also known as B7-H1, CD274) is one of the ligands of PD-1. PD-L1 is broadly expressed on the cell surface of many different immune cell populations (e.g. T-, B- NK-cells, DC, monocytes, macrophages), on activated vascular endothelial cells, but also epithelial cells including tumor cells of various entities such as melanoma, lung, ovarian and colon cancers. The expression of PD-L1 is enhanced by proinflammatory cytokines such as interferon gamma, interferon Type I and gamma chain cytokines (IL-2, -4, -7, -9, -15, -21). As described above, T cell activation is inhibited upon interaction with PD-1 and thereby the immune response is dampened (Park et al., Arch. Pharm. Res. (2016) 39, 1577-1587; Menon et al., Cancers (2016) 8, 106, 1-21).

Several alpha-emitters, such as Terbium-149 (149Tb), Astatine-211 (211At), Bismuth-212 (212Bi), Bismuth-213 (213Bi), Actinium-225 (225Ac), Radium-223 (223Ra), Radium-224 (224Ra), or Thorium-227 (227Th), have been investigated and/or commercialised for use as radiopharmaceuticals. In particular, the use of ‘tissue-targeting’ radiopharmaceuticals has meant that the radioactive nucleus can be delivered to the target cell (for example a cancerous cell) with an improved accuracy, thus minimising unwanted damage to surrounding tissue and hence minimising side effects. Tissue-targeting radiopharmaceuticals are typically conjugates in which the radiopharmaceutical moiety is linked to a targeting unit, for example via a chelator. The targeting unit (for example, an antibody) guides the radiopharmaceutical to the desired cell (by targeting a particular antigen on a cancer cell for example) such that the alpha radiation can be delivered in close proximity to the target. A small number of elements can be considered “self targeting” due to their inherent properties. Radium, for example, is a calcium analogue and targets bone surfaces by this inherent nature.

One particular class of tissue-targeting radiopharmaceuticals is Targeted Thorium Conjugates (TTCs), in which alpha-emitting thorium-227 (Th-227) nuclei are connected to tumor-targeting moieties such as antibodies. The radioactive pharmaceutical exploits the unique properties of elements that emit alpha particles, and the targeting properties of the conjugates help to minimise undesirable side effects.

While considerable advances have been made over the last few years in the field of targeted radiopharmaceuticals, it would be of considerable advantage to provide further targeted therapeutic methods with increased efficiency. In particular, even with efficient targeting, there is a limit to the amount of radionuclide which can be administered to a subject without causing intolerable side-effects such as myelo-suppression. It would be of considerable benefit to provide a therapeutic method or a method of utilising such radionuclides which could enhance the efficacy of the medicament without requiring a higher dose of radiopharmaceutical.

The present inventors have now established that combinations of targeted radiopharmaceuticals with PD-1/PD-L1 inhibitors can improve the therapeutic efficiency of radiopharmaceuticals. In particular, the combination treatment of the present invention may result in an additive, super-additive or synergistic interaction between a radiopharmaceutical and at least one from a range of PD-1/PD-L1 inhibitors and may be employed against various targets and cancer cell lines. A key advantage of the combination therapy of the present invention is the synergistic effect of the PD-1/PD-L1 inhibitors and the tissue-targeting radiopharmaceutical. The PD-1/PD-L1 inhibitors and the tissue-targeting radiopharmaceutical work in tandem to increase the effectiveness in treatment. The combination therapy is thus more effective than the use of the tissue targeting radiopharmaceutical alone or the PD-1/PD-L1 inhibitors alone and the effect of the combination is greater than the sum of the effects of the components used individually.

### SUMMARY OF THE INVENTION

Surprisingly it was observed that by administering a PD-1/PD-L1 inhibitor in combination with a tissue-targeting radiopharmaceutical comprising an alpha-emitter, preferably a complex comprising the 4+ ion of an alpha-emitting thorium radionuclide such as thorium-227, most preferably a targeted thorium conjugate (TTC), a synergistic anti-proliferative and apoptotic effects in prostate, breast and ovarian tumor cell lines.

Therefore, in accordance with a first aspect, the present invention provides combinations of at least two components, component A and component B, component A being a PD-1/PD-L1 inhibitor, and component B being a TTC.

In accordance with a second aspect, the present invention covers combinations of at least two components A and B, component A being a PD-1/PD-L1 inhibitor, and component B being a MSLN-TTC.

In accordance with a third aspect, the present invention comprises combinations of at least two components A and B, component A being a PD-1/PD-L1 inhibitor, and component B being a PSMA-TTC.

In accordance with a third aspect, the present invention comprises combinations of at least two components A and B, component A being a PD-1/PD-L1 inhibitor and component B being a HER2-TTC.

The combinations comprising at least two components A and B, as decribed and defined herein, are also referred to as “combinations of the present invention”.

Further, the present invention relates to:
a kit comprising:
a combination of:

Component A: one or more PD-1/PD-L1 inhibitor(s) as described herein; Component B: a suitable pharmaceutically acceptable TTC; and, optionally, Component C: one or more further pharmaceutical agents;

in which optionally either or both of said components A and B in any of the above-mentioned combinations are in the form of a pharmaceutical formulation/composition which is ready for use to be administered simultaneously, concurrently, separately or sequentially. The components may be administered independently of one another by the oral, intravenous, topical, local installations, intraperitoneal or nasal route.

In accordance with another aspect, the present invention covers the combinations as described supra for the treatment or prophylaxis of a disease.

In accordance with another aspect, the present invention covers the use of such combinations as described supra for the preparation of a medicament for the treatment or prophylaxis of a disease.

### DESCRIPTION OF THE SEVERAL DRAWINGS

FIG. 1: The effects of compound A′ and compound B′ (MSLN-TTC) on OVCAR-3 (A) and OVCAR-8 (B) cells. Presented are the isobolograms as well as the combination index chart from one representative experiment.

FIG. 2: The effects of compound A′ and compound B′ (HER2-TTC) on JIMT-1 (A) and BT-474 (B) cells. Presented are the isobolograms as well as the combination index chart from one representative experiment.

FIG. 3A: The effects of compound A′ and compound B′ (PSMA-TTC) on LNCaP (A) cells. Presented are the isobolograms as well as the combination index chart from one respective experiment.

FIG. 3B: The effects of compound A′ and compound B′ (PSMA-TTC) on 22Rv1 (B), Presented are the isobolograms as well as the combination index chart from one respective experiment.

FIG. 3C: The effects of compound A′ and compound B′ (PSMA-TTC) on MDA-PCa-2b cells (C). Presented are the isobolograms as well as the combination index chart from one respective experiment.

FIG. 3D: The effects of compound A′ and compound B′ (PSMA-TTC) on VCaP (D) cells. Presented are the isobolograms as well as the combination index chart from one respective experiment.

### DETAILED DESCRIPTION OF THE INVENTION

### Definitions

The terms as mentioned in the present text have preferably the following meanings:
The term ‘alkyl’ refers to a straight or branched hydrocarbon chain radical consisting solely of carbon and hydrogen atoms, containing solely of carbon and hydrogen atoms, containing no unsaturation, having from one to eight carbon atoms, and which is attached to the rest of the molecule by a single bond, such as illustratively, methyl, ethyl, n-propyl 1-methylethyl (isopropyl), n-butyl, n-pentyl, and 1,1-dimethylethyl (t-butyl).
The term “alkenyl” refers to an aliphatic hydrocarbon group containing a carbon-carbon double bond and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, 2- and butenyl.
The term “alkynyl” refers to a straight or branched chain hydrocarbonyl radicals having at least one carbon-carbon triple bond, and having in the range of about 2 up to 12 carbon atoms (with radicals having in the range of about 2 up to 10 carbon atoms presently being preferred) e.g., ethynyl.
The term “alkoxy” denotes an alkyl group as defined herein attached via oxygen linkage to the rest of the molecule. Representative examples of those groups are methoxy and ethoxy.
The term “alkoxyakyl” denotes an alkoxy group as defined herein attached via oxygen linkage to an alkyl group which is then attached to the main structure at any carbon from alkyl group that results in the creation of a stable structure the rest of the molecule. Representative examples of those groups are —CH₂OCH₃, —CH₂OC₂H₅.
The term “cycloalkyl” denotes a non-aromatic mono or multicyclic ring system of about 3 to 12 carbon atoms such as cyclopropyl, cyclobutyl, cyclopentyl, cyclohexyl and examples of multicyclic cycloalkyl groups include perhydronapththyl, adamantyl and norbornyl groups bridged cyclic group or sprirobicyclic groups e.g sprio (4,4) non-2-yl.
The term “cycloalkylalkyl” refers to cyclic ring-containing radicals containing in the range of about about 3 up to 8 carbon atoms directly attached to alkyl group which is then also attached to the main structure at any carbon from the alkyl group that results in the creation of a stable structure such as cyclopropylmethyl, cyclobuyylethyl, cyclopentylethyl.
The term “aryl” refers to aromatic radicals having in the range of 6 up to 14 carbon atoms such as phenyl, naphthyl, tetrahydronapthyl, indanyl, biphenyl.
The term “arylalkyl” refers to an aryl group as defined herein directly bonded to an alkyl group as defined herein which is then attached to the main structure at any carbon from alkyl group that results in the creation of a stable structure the rest of the molecule. e.g., —CH₂C₆H₅, —C₂H₅C₆H₅.
The term “heterocyclic ring” refers to a stable 3- to 15 membered ring radical which consists of carbon atoms and from one to five heteroatoms selected from the group consisting of nitrogen, phosphorus, oxygen and sulfur. For purposes of this invention, the heterocyclic ring radical may be a monocyclic, bicyclic or tricyclic ring system, which may include fused, bridged or Spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofurnyl, carbazolyl cinnolinyl dioxolanyl, indolizinyl, naphthyridinyl, perhydroazepinyl, phenazinyl, phenothiazinyl, phenoxazinyl, phthalazil, pyridyl, pteridinyl, purinyl, quinazolinyl, quinoxalinyl, quinolinyl, isoquinolinyl, tetrazoyl, imidazolyl tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl pyridazinyl, oxazolyl oxazolinyl oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide thiamorpholinyl sulfone, dioxaphospholanyl, oxadiazolyl, chromanyl, isochromanyl.
The term “heteroaryl” refers to heterocyclic ring radical as defined herein which are aromatic. The heteroaryl ring radical may be attached to the main structure at any heteroatom or carbon atom that results in the creation of a stable structure.
The heterocyclic ring radical may be attached to the main structure at any heteroatom or carbon atom that results in the creation of a stable structure.
The term “heteroarylalkyl” refers to heteroaryl ring radical as defined herein directly bonded to alkyl group. The heteroarylalkyl radical may be attached to the main structure at any carbon atom from alkyl group that results in the creation of a stable structure.
The term “heterocyclyl” refers to a heterocylic ring radical as defined herein. The heterocylyl ring radical may be attached to the main structure at any heteroatom or carbon atom that results in the creation of a stable structure.
The term “heterocyclylalkyl” refers to a heterocylic ring radical as defined herein directly bonded to alkyl group. The heterocyclylalkyl radical may be attached to the main structure at carbon atom in the alkyl group that results in the creation of a stable structure.
The term “carbonyl” refers to an oxygen atom bound to a carbon atom of the molecule by a double bond.
The term “halogen” refers to radicals of fluorine, chlorine, bromine and iodine.

The term “optionally substituted” means optional substitution with the specified groups, radicals or moieties.

Ring system substituent means a substituent attached to an aromatic or nonaromatic ring system which, for example, replaces an available hydrogen on the ring system.

As used herein, the term “one or more times”, e.g. in the definition of the substituents of the compounds of the present invention (e.g. component A, B or C), is understood as meaning “one, two, three, four or five times, particularly one, two, three or four times, more particularly one, two or three times, even more particularly one or two times”.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The term “carbonyl” refers to an oxygen atom bound to a carbon atom of the molecule by a double bond.

The compounds of this invention may contain one or more asymmetric centers, depending upon the location and nature of the various substituents desired. Asymmetric carbon atoms may be present in the (R)- and/or (S)-configuration, resulting in racemic mixtures in the case of a single asymmetric center, and diastereomeric mixtures in the case of multiple asymmetric centers. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those, which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

Tautomers, sometimes referred to as proton-shift tautomers, are two or more compounds that are related by the migration of a hydrogen atom accompanied by the switch of one or more single bonds and one or more adjacent double bonds. The compounds of this invention may exist in one or more tautomeric forms. For example, a compound of Formula I may exist in tautomeric form Ia, tautomeric form Ib, or tautomeric form Ic, or may exist as a mixture of any of these forms. It is intended that all such tautomeric forms are included within the scope of the present invention.

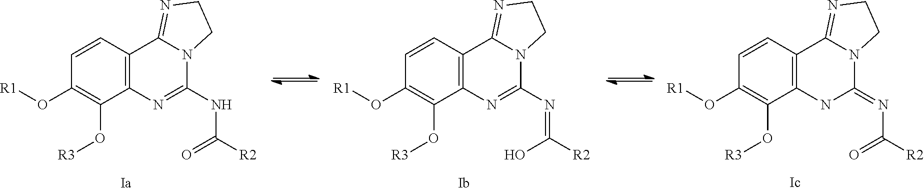

The present invention also relates to useful forms of the compounds as disclosed herein, such as pharmaceutically acceptable salts, co-precipitates, metabolites, hydrates, solvates and prodrugs of all the compounds of examples. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, sulfate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, or butyl chlorides, bromides and iodides; dialkyl sulfates like dimethyl, diethyl, dibutyl sulfate, or diamyl sulfates, long chain halides such as decyl, lauryl, myristyl and strearyl chlorides, bromides and iodides, aralkyl halides like benzyl and phenethyl bromides and others.

A solvate for the purpose of this invention is a complex of a solvent and a compound of the invention in the solid state. Exemplary solvates would include, but are not limited to, complexes of a compound of the invention with ethanol or methanol. Hydrates are a specific form of solvate wherein the solvent is water.

Constituents which are optionally substituted as stated herein, may be substituted, unless otherwise noted, one or more times, independently from one another at any possible position. When any variable occurs more than one time in any constituent, each definition is independent.

The heteroarylic, or heterocyclic groups mentioned herein can be substituted by their given substituents or parent molecular groups, unless otherwise noted, at any possible position, such as e.g. at any substitutable ring carbon or ring nitrogen atom. Analogously it is being understood that it is possible for any heteroaryl or heterocyclyl group to be attached to the rest of the molecule via any suitable atom if chemically suitable. Unless otherwise noted, any heteroatom of a heteroarylic ring with unsatisfied valences mentioned herein is assumed to have the hydrogen atom(s) to satisfy the valences. Unless otherwise noted, rings containing quaternizable amino- or imino-type ring nitrogen atoms (—N═) may be preferably not quaternized on these amino- or imino-type ring nitrogen atoms by the mentioned substituents or parent molecular groups.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques already known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Diacel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

If in the context of the invention “embodiment” is mentioned it should be understood to include a plurality of possible combinations.

In order to limit different types of isomers from each other reference is made to IUPAC Rules Section E (Pure Appl Chem 45, 11-30, 1976).

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

The present invention includes all possible stereoisomers of the compounds of the present invention as single stereoisomers, or as any mixture of said stereoisomers, in any ratio. Isolation of a single stereoisomer, e.g. a single enantiomer or a single diastereomer, of a compound of the present invention may be achieved by any suitable state of the art method, such as chromatography, especially chiral chromatography, for example.

The present invention includes all possible tautomers of the compounds of the present invention as single tautomers, or as any mixture of said tautomers, in any ratio.

Furthermore, the present invention includes all possible crystalline forms, or polymorphs, of the compounds of the present invention, either as single polymorphs, or as a mixture of more than one polymorphs, in any ratio.

### Component a of the Combination

Component B of the combination of the present invention is a PD-1/PD-L1 inhibitor.

The term “PD-1/PD-L1 inhibitor” refers to a PD-1 inhibitor or to a PD-L1 inhibitor.

Particularly, the PD-1 inhibitor is an anti-PD-1 antibody including but not limited to nivolumab (Opdivo, BMS-936558, MDX1106), pembrolizumab (Keytruda, MK-3475, lambrolizumab), PDR-001, JS001, STI-A1110.

Particularly, the PD-L1 inhibitor is an anti-PD-L1 antibody including but not limited to atezolizumab (Tecentriq, MPDL3280A), durvalumab (MEDI4736), avelumab (MSB0010718C), BMS-936559 (MDX1105) and LY3300054.

According to another embodiment of the aspects of the present invention, component A is a “PD-1/PD-L1 inhibitor” selected from nivolumab (Opdivo, BMS-936558, MDX1106), pembrolizumab (Keytruda, MK-3475, lambrolizumab), PDR-001, JS001, STI-A1110, atezolizumab (Tecentriq, MPDL3280A), durvalumab (MEDI4736), avelumab (MSB0010718C), BMS-936559 (MDX1105) and LY3300054.

According to another embodiment of the aspects of the present invention, component A is a “PD-1 inhibitor” selected from nivolumab (Opdivo, BMS-936558, MDX1106), pembrolizumab (Keytruda, MK-3475, lambrolizumab), PDR-001, JS001, STI-A1110.

According to a preferred embodiment of the aspects of the present invention, component A is a PD-1 inhibitor selected from nivolumab and pembrolizumab.

According to a preferred embodiment of the aspects of the present invention, component A is pembrolizumab.

According to another embodiment of the present invention component A is the PD-1 inhibitor RMP1-14.

According to another embodiment of the aspects of the present invention, component A is a “PD-L1 inhibitor” selected from atezolizumab (Tecentriq, MPDL3280A), durvalumab (MEDI4736), avelumab (MSB0010718C), BMS-936559 (MDX1105) and LY3300054.

According to another embodiment of the aspects of the present invention, component A is a PD-L1 inhibitor selected from atezolizumab, durvalumab and avelumab, preferably component B is a PD-L1 inhibitor selected from atezolizumab and avelumab.

According to another embodiment of the aspects of the present invention, component A is atezolizumab.

According to another embodiment of the present invention component B is the PD-L1 inhibitor PPB-6721.

Nivolumab is a human IgG4 anti-PD-1 monoclonal antibody. For example it is used as a first line treatment for inoperable or metastatic melanoma in combination with ipilimumab if the cancer does not have a mutation in BRAF, as a second-line treatment following treatment with ipilimumab and if the cancer has a mutation in BRAF, with a BRAF inhibitor, as a second-line treatment for squamous non-small cell lung cancer, and as a second-line treatment for renal cell carcinoma.

Pembrolizumab is a humanized antibody which is for example indicated
for the treatment of patients with unresectable or metastatic melanoma,
as a single agent for the first-line treatment of patients with metastatic NSCLC whose tumors have high PD-L1 expression [(Tumor Proportion Score (TPS) ≥50%)] as determined by an FDA-approved test, with no EGFR or ALK genomic tumor aberrations,
for the treatment of patients with recurrent or metastatic HNSCC with disease progression on or after platinum-containing chemotherapy.

PDR-001 is an intravenously administered anti-PD-1 antibody. In July 2017, Phase III trials for malignant melanoma, Phase II trials for nasopharyngeal cancer and for neuroendocrine tumors and Phase I/II trials for solid tumors and Phase I trials for hepatocellular carcinoma, lymphoma and colorectal cancer are ongoing.

JS001 is a recombinant humanised monoclonal antibody. Phase II development for melanoma and bladder cancer, Phase I/II trial for gastric cancer, nasopharyngeal cancer, oesophageal cancer and head and neck cancer and Phase I development in breast cancer, lymphoma, urogenital cancer, renal cancer, neuroendocrine tumors and solid tumors are ongoing in July 2017.

STI-A1110 is a lead monoclonal antibody (MAb) against programmed cell death protein 1 (PD-1), under development by Sorrento Therapeutics using its G-MAB fully human antibody library platform, for the treatment of cancer (Company presentation, Sorrento, 13 Mar. 2017, Slide 10, http://sorrentotherapeutics.com/wp-content/uploads/2017/03/Sorrento-Corporate-Presentation-ROTH-Mar-2017-FINAL.pdf; Company Web Page, Sorrento, 19 May 2017, http://sorrentotherapeutics.com/platforms/immuno-oncology-antibodies/). An initiation of clinical trial is expected in 2H 2017 (Company presentation, Sorrento, 1 Nov. 2016, Slide 7, http://sorrentotherapeutics.com/wp-content/uploads/2016/11/Sorrento-Corporate-Presentation-JefConf-FINAL.pdf).

Atezolizumab is a programmed death-ligand 1 (PD-L1) blocking antibody indicated for the treatment of patients with locally advanced or metastatic urothelial carcinoma who
have disease progression during or following platinum-containing chemotherapy.
have disease progression within 12 months of neoadjuvant or adjuvant treatment with platinum-containing chemotherapy.

Atezolizumab is also indicated for the treatment of patients with metastatic non-small cell lung cancer who have disease progression during or following platinum-containing chemotherapy. Patients with EGFR or ALK genomic tumor aberrations should have disease progression on FDA-approved therapy for these aberrations prior to receiving Atezolizumab.

Durvalumab is a PD-L1 blocking antibody indicated for the treatment of patients with locally advanced or metastatic urothelial carcinoma who:
have disease progression during or following platinum-containing chemotherapy.
have disease progression within 12 months of neoadjuvant or adjuvant treatment with platinum-containing chemotherapy.

Avelumab is a PD-L1 blocking antibody indicated for the treatment of adults and pediatric patients 12 years and older with metastatic Merkel cell carcinoma (MCC).

BMS-936559 is a PD-L1 blocking antibody.

LY3300054 is a PD-L1 blocking antibody. Phase I development in solid tumors, Microsatellite Instability-High (MSI-H) solid tumors and in cutaneous melanoma are ongoing in July 2017.

Component A may be administered by the oral, intravenous, topical, local installations, intraperitoneal or nasal route.

Component A may be in the form of a pharmaceutical formulation which is ready for use to be administered simultaneously, concurrently, separately or sequentially with component A and optionally component C as further described infra. The components A and B and optionally C may be administered independently of one another by the oral, intravenous, topical, local installations, intraperitoneal or nasal route.

The PD-1/PD-L1 inhibitor mentioned in the prior art as well as in the lists above have been disclosed for the treatment or prophylaxis of different diseases, especially cancer.

The specific compounds of the lists as disclosed above are preferred as being component A of the combination, most preferred is the compound used in the experimental section.

The synergistic behavior of a combination of the present invention is demonstrated herein with one of the PD-1/PD-L1 inhibitor specifically disclosed in the Examples section as compound A.

In addition a combination of the present invention comprising compound A as mentioned above and a targeted thorium conjugate is a preferred aspect of the invention.

In another aspect a combination of the present invention comprises compound A or a pharmaceutically acceptable salt thereof as mentioned above and a targeted thorium conjugate selected from the list consisting of PSMA-TTC, HER2-TTC and MSLN-TTC.

It is to be understood that the present invention relates also to any combination of the embodiments of component A described above.

### Component B of the Combination

Component B is a suitable tissue targeting radiopharmaceutical.

In the context of the present invention, “tissue targeting” is used herein to indicate that the substance in question (particularly when in the form of a tissue-targeting complex as described herein), serves to localise itself (and particularly to localise any conjugated thorium complex) preferentially to at least one tissue site at which its presence is desired (e.g. to deliver a radioactive decay). Thus a tissue targeting group or moiety serves to provide greater localisation of a radioisotope to at least one desired site in the body of a subject following administration to that subject in comparison with the concentration of an equivalent radioisotope or complex not bound to the targeting moiety. The targeting moiety in the present case will be preferably selected to bind specifically to cell-surface targets (e.g. receptors) associated with cancer cells or other targets associated with the tumour microenvironment. There are a number of targets which are known to be associated with hyperplastic and neoplastic disease. These include certain receptors, cell surface proteins, transmembrane proteins and proteins/peptides found in the extracellular matrix in the vicinity of diseased cells.

Tissue-targeting radiopharmaceuticals of the various aspects of the present invention preferably comprise a tissue-targeting moiety. Such a moiety may be, for example, an antibody or antibody derivative, such as one selected from a monoclonal or polyclonal antibody, an antibody fragment (such as Fab, F(ab′)2, Fab′ or scFv), or a construct of such antibodies and/or fragments. Mixtures of such antibodies and/or derivatives are evidently also appropriate. Some examples of engineered antibodies are listed herein below.

The targeting moiety is preferably tumour-homing, i.e. it targets cancer cells. Such cancer cell targeting is typically the result of the targeting moiety targeting a tumour-associated antigen. In one embodiment, therefore, the tissue targeting moiety may bind to a tumour-associated antigen. Many such tumour associated antigens are known in the art, including “Cluster of Differentiation (CD)” antigens (e.g. CD20, CD22, CD30, CD32, CD33 and/or CD52), glycoprotein antigens (e.g. EpCAM, CEA, Mucins, TAG-72m Carbonic anhydrase IX, PSMA and/or folate binding protein), Glycolipid antigens (e.g. Gangliosides such as GD2, GD3, and/or GM2), Carbohydrate antigens (e.g. Lewis-Y), Vascular antigens (e.g. VEGF, VEGFR, αVβ3, α5β1), Growth factor antigens (e.g. ErbB1, EGFR, ErbB2, HER2, ErbB3, c-MET, IGF1R, EphA3, TRAIL-R!, TRAIL-R2, RANKL), extracellular matrix antigens (e.g. FAP, Tenascin), and/or overexpressed receptors (e.g αᵥβ₃).

The antibody may be an antibody (e.g. a monoclonal antibody) which is in itself an immunotherapeutic agent which binds to certain cells or proteins and then stimulates the patient's immune system to attack those cells. In this case, the radiopharmaceutical acts in tandem with the immunotherapeutic effects of the antibody. Alternatively, the targeting moiety may act solely as a targeting agent and does not provoke any immunotherapeutic effects by itself. In this case, it is solely the radiopharmaceutical unit which acts as the active, cell-destroying agent, supported in the combination therapy methods of the present invention by at least one DNA repair inhibitor.

In one embodiment, the tissue-targeting radiopharmaceutical may comprise a tissue-targeting moiety selected from at least one engineered antibody. Such an engineered antibody may be an antibody that comprises an epitope binding domain (for example, but not limited to, an antibody variable region having all 6 CDRs, or an equivalent region that is at least 90% identical to an antibody variable region) chosen from: abagovomab, abatacept (also known as ORENCIA®), abciximab (also known as REOPRO®, c7E3 Fab), adalimumab (also known as HUMIRA®), adecatumumab, alemtuzumab (also known as CAMPATH®, MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT®), bavituximab, bectumomab (also known as LYMPHOSCAN®), belimumab (also known as LYMPHO-STAT-B®), bertilimumab, besilesomab, bevacizumab (also known as AVASTIN®), biciromab brallobarbital, bivatuzumab mertansine, campath, canakinumab (also known as ACZ885), cantuzumab mertansine, capromab (also known as PROSTASCINT®), catumaxomab (also known as REMOVAB®), cedelizumab (also known as CIMZIA®), certolizumab pegol, cetuximab (also known as ERBITUX®), clenoliximab, dacetuzumab, dacliximab, daclizumab (also known as) ZENAPAX®), denosumab (also known as AMG 162), detumomab, dorlimomab aritox, dorlixizumab, duntumumab, durimulumab, durmulumab, ecromeximab, eculizumab (also known as SOLIRIS®), edobacomab, edrecolomab (also known as Mab17-1A, PANOREX®), efalizumab (also known as RAPTIVA®), efungumab (also known as MYCOGRAB®), elsilimomab, enlimomab pegol, epitumomab cituxetan, efalizumab, epitumomab, epratuzumab, erlizumab, ertumaxomab (also known as REXOMUN®), etanercept (also known as ENBREL®), etaracizumab (also known as etaratuzumab, VITAXIN®, ABEGRINT™), exbivirumab, fanolesomab (also known as NEUTROSPEC®), faralimomab, felvizumab, fontolizumab (also known as HUZAF®), galiximab, gantenerumab, gavilimomab (also known as ABX-CBL®), gemtuzumab ozogamicin (also known as MYLOTARG®), golimumab (also known as CNTO 148), gomiliximab, ibalizumab (also known as TNX-355), ibritumomab tiuxetan (also known as ZEVALIN®), igovomab, imciromab, infliximab (also known as REMICADE®), inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-ST01), lexitumumab, libivirumab, lintuzumab, lucatumumab, lumiliximab, mapatumumab (also known as HGS-ETR1, TRM-1), maslimomab, matuzumab (also known as EMD72000), mepolizumab (also known as BOSATRIA®), metelimumab, milatuzumab, minretumomab, mitumomab, morolimumab, motavizumab (also known as NUMAXT™), muromonab (also known as OKT3), nacolomab tafenatox, naptumomab estafenatox, natalizumab (also known as TYSABRI®, ANTEGREN®), nebacumab, nerelimomab, nimotuzumab (also known as THERACIM hR3®, THERA-CIM-hR3®, THERALOC®), nofetumomab merpentan (also known as VERLUMA®), ocrelizumab, odulimomab, ofatumumab, omalizumab (also known as XOLAIR®), oregovomab (also known as OVAREX®), otelixizumab, pagibaximab, palivizumab (also known as SYNAGIS®), panitumumab (also known as ABX-EGF, VECTIBIX®), pascolizumab, pemtumomab (also known as THERAGYN®), pertuzumab (also known as 2C4, OMNITARG®), pexelizumab, pintumomab, priliximab, pritumumab, ranibizumab (also known as LUCENTIS®), raxibacumab, regavirumab, reslizumab, rituximab (also known as RITUXAN®, MabTHERA®), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN®), tacatuzumab tetraxetan, tadocizumab, talizumab, taplitumomab paptox, tefibazumab (also known as AUREXIS®), telimomab aritox, teneliximab, teplizumab, ticilimumab, tocilizumab (also known as ACTEMRA®), toralizumab, tositumomab, trastuzumab (also known as HERCEPTIN®), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION®), volociximab (also known as M200), votumumab (also known as HUMASPECT®), zalutumumab, zanolimumab (also known as HuMAX-CD4), ziralimumab, or zolimomab aritox.

While antibodies as tissue-targeting moiety constitute a preferred embodiment of the invention, the targeting unit may also be a single type of protein, protein fragment or construct of protein, or a mixture of proteins, fragments or constructs of protein. Where peptides are referred to herein, corresponding peptidomimetics may also be utilised. Combinations of targeting moieties of any type may also be used.

The targeting moiety may also be a peptide such as Tat-peptide, penetratin, MPG and Pep-1. Protein fragments, such as histidine-rich glycoprotein fragments, for example HRGP-335 also constitute an embodiment of the invention. Tumor-homing peptides such as the NGR- and cRGD peptides constitute a further embodiment. Suitable moieties also include other poly- and oligo-peptides including peptidomemetics.

The targeting moiety may also be a small molecule ligand. By small molecule ligand is meant a ligand of low molecular weight, for example having a molecular weight of less than 1000 g/mol (e.g. 50 to 1000), preferably less than 500 or less than 250 g/mol. In particular, the targeting moiety may be a PSMA-targeting ligand. Of particular interest are ligands targeting the enzymatic binding pocket derived from either phosphonate, phosphate and phosphoramidates, thiols and ureas. Suitable PSMA ligands may, for example, comprise at least one moiety selected from a carbon-sulfur double bond, a phosphorus-sulfur double bond, a phosphorus-sulfur single bond, a thioester, a phosphonate, a phosphate, a phosphoramidate, a thiol, and/or a urea.

It is also envisaged that aptamers, DNA or RNA fragments may be used as targeting moieties in the present invention.

Surface-modified nanoparticles that include, but are not limited to, liposomes, nanoworms, and dendrimers may also be used as the targeting unit and thus constitute a further embodiment of the invention.

Examples of cell-surface receptors and antigens which may be associated with neoplastic disease include CD22, CD33, FGFR2 (CD332), PSMA, HER2, Mesothelin etc. Therefore, in a particularly preferred embodiment of the invention, the tissue-targeting moiety (e.g. peptide or protein) has specificity for at least one antigen or receptor selected from CD22, CD33, FGFR2 (CD332), PSMA, HER2 and Mesothelin.

CD22, or cluster of differentiation-22, is a molecule belonging to the SIGLEC family of lectins (SIGLEC=Sialic acid-binding immunoglobulin-type lectins). CD33 or Siglec-3 is a transmembrane receptor expressed on cells of myeloid lineage. FGFR2 is a receptor for fibroblast growth factor. It is a protein that in humans is encoded by the FGFR2 gene residing on chromosome 10. HER2 is a member of the human epidermal growth factor receptor (HER/EGFR/ERBB) family. Prostate-specific membrane antigen (PSMA) is an enzyme that in humans is encoded by the FOLH1 (folate hydrolase 1) gene. Mesothelin, also known as MSLN, is a protein that in humans is encoded by the MSLN gene.

One tissue-targeting binder in the present case will be selected to bind specifically to CD22 receptor. This may be reflected, for example by having 50 or more times greater binding affinity for cells expressing CD22 than for non-CD22 expressing cells (e.g. at least 100 time greater, preferably at least 300 times greater). It is believed that CD22 is expressed and/or over-expressed in cells having certain disease states (as indicated herein) and thus the CD22 specific binder may serve to target the complex to such disease-affected cells. Similarly a tissue targeting moiety may bind to cell-surface markers (e.g. CD22 receptors) present on cells in the vicinity of disease affected cells. CD22 cell-surface markers may be more heavily expressed on diseased cell surfaces than on healthy cell surfaces or more heavily expressed on cell surfaces during periods of growth or replication than during dormant phases. In one embodiment, a CD22 specific tissue-targeting binder may be used in combination with another binder for a disease-specific cell-surface marker, thus giving a dual-binding complex. Tissue-targeting binders for CD-22 will typically be peptides or proteins, as discussed herein. The various aspects of the invention as described herein relate to treatment of disease, particularly for the selective targeting of diseased tissue, as well as relating to complexes, conjugates, medicaments, formulation, kits etc. useful in such methods. In all aspects, the diseased tissue may reside at a single site in the body (for example in the case of a localised solid tumour) or may reside at a plurality of sites (for example where several joints are affected in arthritis or in the case of a distributed or metastasised cancerous disease).

Other ligands particularly suitable for various embodiments applicable to all aspects of the invention include PSMA ligands for use in prostate cancer, HER2 ligands for use in breast and gastric cancer, and Mesothelin ligands for use in mesothelioma, ovarian, lung and pancreatic cancers. Suitable ligands/binders for each of these targets are known in the art and may be applied using the methods described herein.

Radioactive Nuclei

The tissue-targeting radiopharmaceutical preferably comprises an alpha-emitter. The radioactive isotope may be any alpha-emitting isotope (i.e. an alpha emitter) suitable for use in the treatments of the present invention. The alpha emitters may be selected from the group consisting of Terbium-149 (¹⁴⁹Th), Astatine-211 (²¹¹At) Bismuth-212 (²¹²Bi), Bismuth-213 (²¹³Bi), Actinium-225 (²²⁵Ac), or Thorium-227 (²²⁷Th). Preferably, the alpha-emitting nucleus is Thorium-227.

In one embodiment of the present invention, the alpha-emitting radioisotope is not Radium 223 (²²³Ra) or Radium-224 (²²⁴Ra). It is particularly preferable that the alpha-emitting radioisotope is not Radium-223 (²²³Ra). In such an embodiment, it is preferred that the radiopharmaceutical comprises an alpha-emitting radioisotope other than Radium-223. In a corresponding embodiment, the radiopharmaceutical does not comprise any Radium-223 or includes ²²³Ra only as a decay product and/or unavoidable impurity. In a further embodiment, it is preferably if the alpha-emitting radioisotope can be complexed and/or conjugated to ligands.

In a particular embodiment of the invention the tissue-targeting radiopharmaceutical is a complex comprising the 4+ ion of an alpha emitting thorium radionuclide, such as Thorium-227. Preferably, the tissue-targeting radiopharmaceutical is a targeted thorium conjugate (TTC). The targeted thorium conjugate may be any conjugate which comprises an alpha-radioactive thorium ion (e.g. Thorium-227 ion) linked to a targeting moiety such as those described previously. In particular, preferred targeted thorium conjugates include MSLN-TTC, FGFR2-TTC, HER2-TTC, PSMA-TTC, and CD22-TTC.

Radioactive thorium-containing compounds (e.g. comprising Th-227) may be used in high dose regimens, where the myelotoxicity of the generated radium (e.g. Ra-223) would normally be intolerable, when stem cell support or a comparable recovery method is included. Without supportive intervention, the maximum dose of a nuclide such as ²²⁷Th may be limited by such myelotoxicity and might be stopped, for example, to avoid depressing the neutrophil cell count below 20% or 10% of its initial value at nadir. In cases of stem-cell support or similar supportive therapy is provided, the neutrophil cell count may be reduced to below 10% at nadir and exceptionally will be reduced to 5% or if necessary below 5%, providing suitable precautions are taken and subsequent stem cell support is given. Such techniques are well known in the art.

Alpha-emitting thorium is the preferred radioactive element comprised in the tissue-targeting radiopharmaceuticals referred to herein and Thorium-227 is the preferred isotope for all references to thorium herein where context allows. Thorium-227 is relatively easy to produce and can be prepared indirectly from neutron irradiated Ra-226, which will contain the mother nuclide of Th-227, i.e. Ac-227 (T1/2=22 years). Actinium-227 can quite easily be separated from the Ra-226 target and used as a generator for Th-227. This process can be scaled to industrial scale if necessary, and hence the supply problem seen with most other alpha-emitters considered candidates for molecular targeted radiotherapy can be avoided. Thorium-227 decays via radium-223. In this case the primary daughter has a half-life of 11.4 days. From a pure Th-227 source, only moderate amounts of radium are produced during the first few days. However, the potential toxicity of Ra-223 is higher than that of Th-227 since the emission from Ra-223 of an alpha particle is followed within minutes by three further alpha particles from the short-lived daughters.

Partly because it generates potentially harmful decay products, thorium-227 (T1/2=18.7 days) has not been widely considered for alpha particle therapy.

Thorium-227 may be administered in amounts sufficient to provide desirable therapeutic effects without generating so much radium-223 as to cause intolerable bone marrow suppression. It is desirable to maintain the daughter isotopes in the targeted region so that further therapeutic effects may be derived from their decay. However, it is not necessary to maintain control of the thorium decay products in order to have a useful therapeutic effect without inducing unacceptable myelotoxicity. Without being bound by theory, this is believed to be because at least partial incorporation of the radium-223 into bone and the short half-life of the daughters serves to titrate the potentially harmful daughter nuclei away from sensitive structures such as the bone marrow.

The alpha-emitting isotope of the radiopharmaceutical may be linked to the tissue-targeting moiety via any suitable ligand. Such a ligand will be selected to be appropriate for the chemistry of the relevant element and oxidation state and suitable chelators are generally well-known in the art.

Previously known chelators for thorium, for example, include the polyaminopolyacid chelators which comprise a linear, cyclic or branched polyazaalkane backbone with acidic (e.g. carboxyalkyl) groups attached at backbone nitrogens. Examples of such chelators include DOTA derivatives such as p-isothiocyanatobenzyl-1,4,7,10-tetraazacyclododecane-1,4,7,10-tetraacetic acid (p-SCN-Bz-DOTA) and DTPA derivatives such as p-isothiocyanatobenzyl-diethylenetriaminepentaacetic acid (p-SCN-Bz-DTPA), the first being cyclic chelators, the latter linear chelators.

In one particular embodiment of the invention, the tissue-targeting radiopharmaceutical comprises a tissue-targeting moiety covalently bound to an octadentate ligand, examples of which include ligands comprising at least one 3,2-hydroxypyridinone (3,2-HOPO) moiety. Said ligand may be complexed to a 4+ metal ion such as that of and alpha-emitting thorium radionuclide (e.g. ²²⁷Th). Such ligands are described, for example, in WO2011/098611 which is incorporated herein by reference. The ligand may therefore be an octadentate ligand, particularly an octadentate hydroxypyridinone-containing ligand. Such ligands will typically comprise at least one chelating group of the following substituted pyridine structure (I):

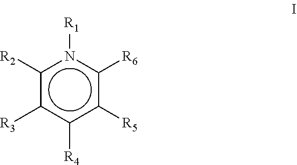

Wherein R₁ is an optional N-substituent group and may thus be absent or may be selected from hydrocarbyl, OH, O-hydrocarbyl, SH and S-hydrocarbyl groups (e.g. methyl or ethyl); comprises a linker moiety; and/or comprises a coupling moiety; groups R2 to R6 are each independently selected from H, OH, ═O, short hydrocarbyl groups (e.g. methyl, ethyl, propyl), linker moieties (linking to other moieties of formula I) and/or coupling moieties (coupling to targeting agents). Favoured ligands may have four moieties of formula I as described in WO2011/098611. Particular examples include octadentate 3,2-HOPO ligands such as those indicated below, as well as equivalent ligands additionally substituted with linker groups (if needed), as discussed herein:

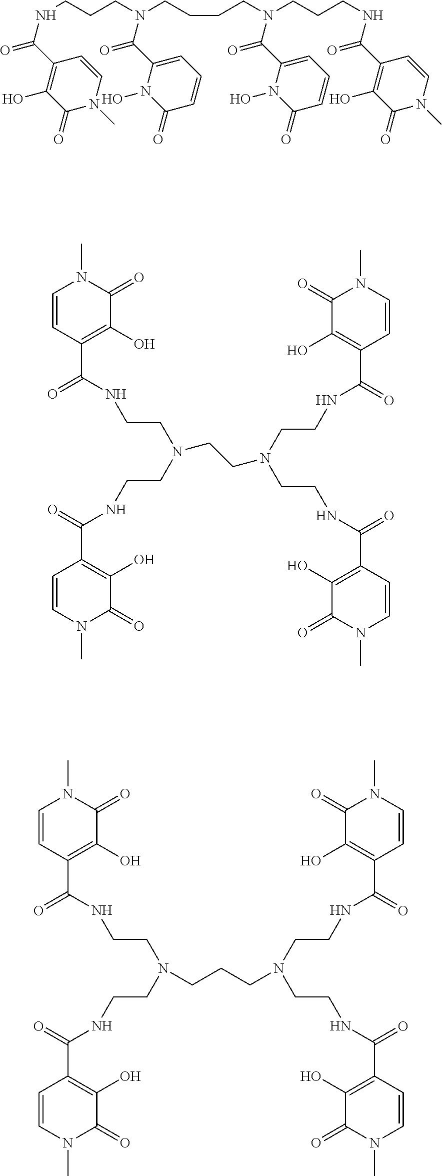 [SMILES: Cn1ccc(C(=O)NCCCN(CCCCN(CCCNC(=O)c2ccn(C)c(=O)c2O)C(=O)c2cccc(=O)n2O)C(=O)c2cccc(=O)n2O)c(O)c1=O ; Cn1ccc(C(=O)NCCN(CCNC(=O)c2ccn(C)c(=O)c2O)CCN(CCNC(=O)c2ccn(C)c(=O)c2O)CCNC(=O)c2ccn(C)c(=O)c2O)c(O)c1=O ; Cn1ccc(C(=O)NCCN(CCCN(CCNC(=O)c2ccn(C)c(=O)c2O)CCNC(=O)c2ccn(C)c(=O)c2O)CCNC(=O)c2ccn(C)c(=O)c2O)c(O)c1=O]

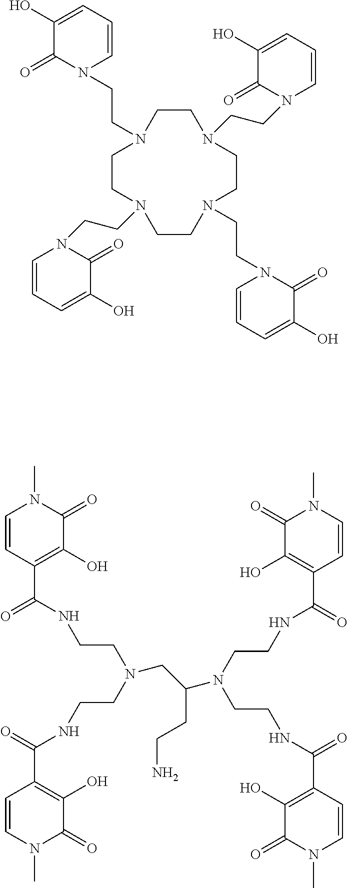 [SMILES: O=c1c(O)cccn1CCN1CCN(CCn2cccc(O)c2=O)CCN(CCn2cccc(O)c2=O)CCN(CCn2cccc(O)c2=O)CC1 ; Cn1ccc(C(=O)NCCN(CCNC(=O)c2ccn(C)c(=O)c2O)CC(CCN)N(CCNC(=O)c2ccn(C)c(=O)c2O)CCNC(=O)c2ccn(C)c(=O)c2O)c(O)c1=O]

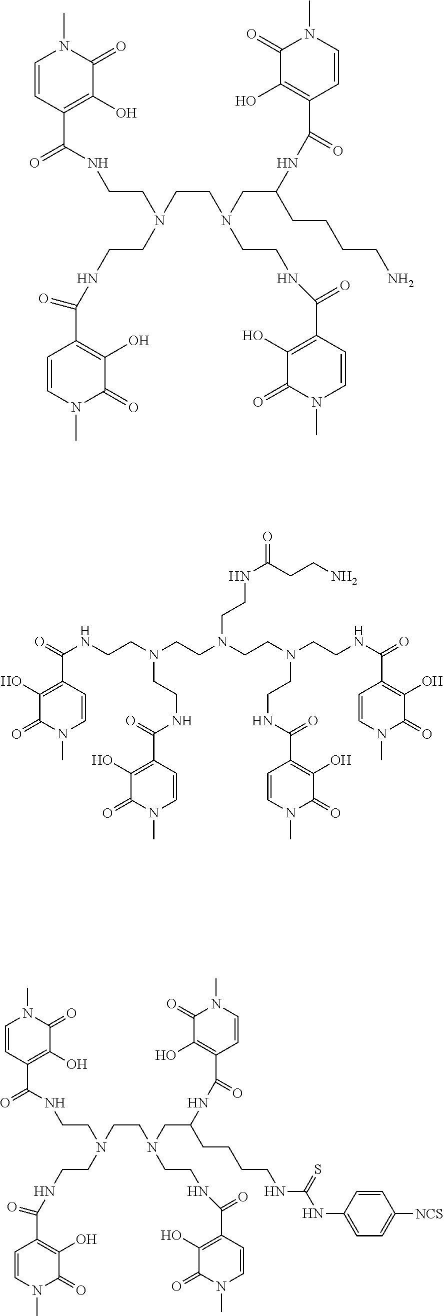

An alternative favoured embodiment utilises ligands as described in WO2013/167756, which is incorporated herein by reference. Such ligands may also be complexed to a 4+ metal ion such as that of an alpha-emitting thorium radionuclide (e.g. ²²⁷Th). In such a particular embodiment, the ligand can be an octadentate ligand comprising at least one and preferably two or four chelating moieties of formula II:

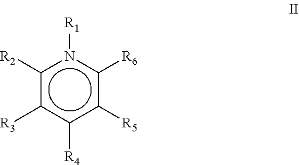

Wherein R₁ is an optional N-substituent solubilising group which will be present in at least one of the moieties of formula II (e.g. in 1 to 4 of four moieties of formula II) and comprises a hydroxyalkyl group (e.g. hydroxymethyl or hydroxydethyl group); groups R₂ to R₆ are each independently selected from H, OH, ═O, short hydrocarbyl groups, linker moieties and/or coupling moieties wherein one of R₂ to R₆ is OH and one of R₂ to R₆ is ═O. The remaining groups R₂ to R₆ may be as described above. The ligand may for example be a ligand of structure III:

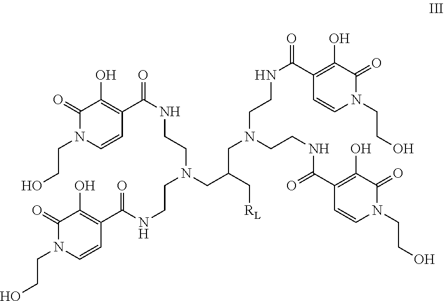

Wherein R_{L} is any suitable linker moiety such as -Ph-NH₂, -Ph-NCS, -Ph-NH—CO—C₂H₄—CO₂H or any described herein.

As used herein, the term “linker moiety” is used to indicate a chemical entity which serves to join at least two chelating groups in the octadentate ligands, which form a key component in various aspects of the invention. Typically, each chelating group (e.g. those of formula I above and/or formula II below) will be bi-dentate and so four chelating groups, of which at least one is of formula I, will typically be present in the ligand. Such chelating groups are joined to each other by means of their linker moieties. Thus, a linker moiety (as used above) may be shared between more than one chelating group of formula I and/or II. The linker moieties may also serve as the point of attachment between the complexing part and the targeting moiety. In such a case, at least one linker moiety will join to a coupling moiety (see below). Suitable linker moieties include short hydrocarbyl groups, such as C1 to C12 hydrocarbyl, including C1 to C12 alkyl, alkenyl or alkynyl group, including methyl, ethyl, propyl, butyl, pentyl and/or hexyl groups of all topologies.

Linker moieties may also be or comprise any other suitably robust chemical linkages including esters, ethers, amine and/or amide groups. The total number of atoms joining two chelating moieties (counting by the shortest path if more than one path exists) will generally be limited, so as to constrain the chelating moieties in a suitable arrangement for complex formation. Thus, linker moieties will typically be chosen to provide no more than 15 atoms between chelating moieties, preferably, 1 to 12 atoms, and more preferably 1 to 10 atoms between chelating moieties. Where a linker moiety joins two chelating moieties directly, the linker will typically be 1 to 12 atoms in length, preferably 2 to 10 (such as ethyl, propyl, n-butyl etc). Where the linker moiety joins to a central template (see below) then each linker may be shorter with two separate linkers joining the chelating moieties. A linker length of 1 to 8 atoms, preferably 1 to 6 atoms may be preferred in this case (methyl, ethyl and propyl being suitable, as are groups such as these having an ester, ether or amide linkage at one end or both).

A “coupling moiety” as used herein serves to link the ligand component (e.g. with 4 moieties of formula I and/or II) to the targeting moiety. Preferably coupling moieties will be covalently linked to the chelating groups, either by direct covalent attachment to one of the chelating groups or more typically by attachment to a linker moiety or template. Should two or more coupling moieties be used, each can be attached to any of the available sites such as on any template, linker or chelating group.

In one embodiment, the coupling moiety may have the structure:

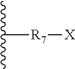

wherein R₇ is a bridging moiety, which is a member selected from substituted or unsubstituted alkyl, substituted or unsubstituted heteroalkyl, substituted or unsubstituted heterocycloalkyl, substituted or unsubstituted aryl and substituted or unsubstituted heteroaryl; and X is a targeting moiety or a reactive functional group. The preferred bridging moieties include all those groups indicated herein as suitable linker moieties. Preferred targeting moieties include all of those described herein and preferred reactive X groups include any group capable of forming a covalent linkage to a targeting moiety, including, for example, COOH, OH, SH, NHR and COH groups, where the R of NHR may be H or any of the short hydrocarbyl groups described herein. Highly preferred groups for attachment onto the targeting moiety include epsilon-amines of lysine residues and thiol groups of cysteine residues. Non-limiting examples of suitable reactive X groups, include N-hydroxysuccimidylesters, imidoesters, acylhalides, N-maleimides, alpha-halo acetyl and isothiocyanates, where the latter three are suitable for reaction with a thiol group.

Another typical example of an octadentate chelator suitable for use in the present invention is the compound of formula IV below, which utilises the 3-hydroxy-N-methyl-2-pyridinone moiety, abbreviated as Me-3,2-HOPO.

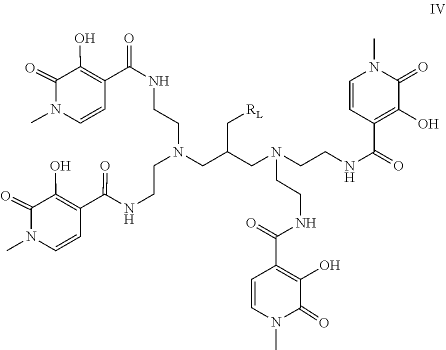

In a particularly favoured embodiment, R_{L} may be such that formula IV is the compound of formula IV′:

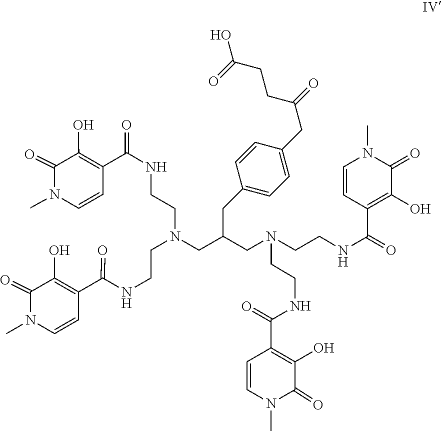 [SMILES: Cn1ccc(C(=O)NCCN(CCNC(=O)c2ccn(C)c(=O)c2O)CC(Cc2ccc(CC(=O)CCC(=O)O)cc2)CN(CCNC(=O)c2ccn(C)c(=O)c2O)CCNC(=O)c2ccn(C)c(=O)c2O)c(O)c1=O]

This particular chelator (IV′) has been found to complex Th-227 in near quantitative yield at ambient temperature in aqueous solutions, and the resulting complexes are highly stable. The carboxylic acid group facilitates conjugation to biomolecules such as antibodies. The synthesis, labelling and in vivo distribution in mice are described in: Bioorganic & Medicinal Chemistry Letters 26 (2016) 4318-4321. It has been shown that the above compound IV′ outperforms 1,4,7,10-tetraazacycloododecane-N, N′,N″,N′″-tetraacetic acid (DOTA) in Th-227 complexation.

In one embodiment, MSLN-TTC is BAY2287411 and is prepared according to Example 7, specifically Examples 7a and 7b of WO 2016/096843.

In one embodiment, FGFR2-TTC is BAY2304058 and is prepared according to Example 6, specifically Examples 6a and 6b of WO 2016/096843.

In one embodiment, HER2-TTC is BAY 2331370 and is prepared according to Example 3, particularly Examples 3.1-3.4 of WO 2017/162555.

In one embodiment, PSMA-TTC is BAY 2315497 and is prepared according to Example 9, specifically Examples 9a and 9b of WO 2016/096843. The monoclonal antibody may be AB-PG1-XG1-006 as disclosed in WO 03/034903.

In all aspects of the present invention, the tissue-targeting radiopharmaceutical preferably comprises Th-227. The radiopharmaceutical is preferably administered at a dosage level of thorium-227 dosage of 18 to 400 kBq/kg bodyweight, preferably 20 to 200 kBq/kg, (such as 50 to 200 kBq/kg) more preferably 75 to 170 kBq/kg, especially 100 to 130 kBq/kg. Correspondingly, a single dosage until may comprise around any of these ranges multiplied by a suitable bodyweight, such as 30 to 150 Kg, preferably 40 to 100 Kg (e.g. a range of 540 kBq to 4000 KBq per dose etc). The thorium dosage, the complexing agent and the administration route will moreover desirably be such that the radium-223 dosage generated in vivo is less than 300 kBq/kg, more preferably less than 200 kBq/kg, still more preferably less than 150 kBq/kg, especially less than 100 kBq/kg. Again, this will provide an exposure to Ra-223 indicated by multiplying these ranges by any of the bodyweights indicated. The above dose levels are preferably the fully retained dose of Th-227 but may be the administered dose taking into account that some Th-227 will be cleared from the body before it decays.

Where the biological half-life of the Th-227 complex is short compared to the physical half-life (e.g. less than 7 days, especially less than 3 days) significantly larger administered doses may be needed to provide the equivalent retained dose. Thus, for example, a fully retained dose of 150 kBq/kg is equivalent to a complex with a 5 day half-life administered at a dose of 711 kBq/kg. The equivalent administered dose for any appropriate retained doses may be calculated from the biological clearance rate of the complex using methods well known in the art.

In accordance with an embodiment, the present invention relates to a combination of any component A mentioned herein with any component B mentioned herein, optionally with any component C mentioned herein.

In one embodiment component A of the combination is the compound used in the experimental section and Component B is a targeted thorium conjugate as being used in the experimental section.

In a particular embodiment, the present invention relates to a combination of a component A with a component B, optionally with a component C, as mentioned in the Examples Section herein.

Further, the present invention relates to:
a kit comprising:
a combination of:
component A: one or more PD-1/PD-L1 inhibitors;
component B: targeted thorium conjugate; and, optionally,
component C: one or more further pharmaceutical agents;
   in which optionally either or both of said components A and B in any of the above-mentioned combinations are in the form of a pharmaceutical formulation which is ready for use to be administered simultaneously, concurrently, separately or sequentially.

The term “component C” being at least one pharmaceutical agent includes the effective compound itself as well as its pharmaceutically acceptable salts, solvates, hydrates or stereoisomers as well as any composition or pharmaceutical formulation comprising such effective compound or its pharmaceutically acceptable salts, solvates, hydrates or stereoisomers. A list of such readily available agents is being provided further below.

The components may be administered independently of one another by the oral, intravenous, topical, local installations, intraperitoneal or nasal route.

Component A is administered intravenously, intraperitoneally, preferably it is administered orally.

Component B preferably is administered by the intravenous route.

Component C being administered as the case may be.

The term “pharmaceutically acceptable” is used synonymously to the term “physiologically acceptable”.

The term “pharmaceutically or physiologically acceptable salt” of component A refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

Representative salts of a component A of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, sulfate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, or butyl chlorides, bromides and iodides; dialkyl sulfates like dimethyl, diethyl, dibutyl sulfate, or diamyl sulfates, long chain halides such as decyl, lauryl, myristyl and strearyl chlorides, bromides and iodides, aralkyl halides like benzyl and phenethyl bromides and others.

A solvate for the purpose of this invention is a complex of a solvent and a compound of the invention in the solid state. Exemplary solvates would include, but are not limited to, complexes of a compound of the invention with ethanol or methanol. Hydrates are a specific form of solvate wherein the solvent is water.

Components of this invention can be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

Components of this invention can also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions can be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs can be formulated with sweetening agents such as, for example, glycerol, propylene glycol, sorbitol or sucrose. Such formulations may also contain a demulcent, and preservative, such as methyl and propyl parabens and flavoring and coloring agents.

Components of this invention can also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions can be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation can also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

Components of the invention can also be administered in the form of suppositories for rectal administration of the drug. These components can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It can be desirable or necessary to introduce a component of the present invention to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, crosslinked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile IV Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular suspension: The following solution or suspension can be prepared, for intramuscular injection:

50 mg/mL of the desired, water-insoluble compound of this invention

5 mg/mL sodium carboxymethylcellulose

4 mg/mL TWEEN 80

9 mg/mL sodium chloride

9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Commercial Utility

Component A

Due to the mechanism as discussed in the introductory section component A is especially suitable to have effects on tumor diseases.

Component B

Due to the mechanism as discussed in the introductory section component B is especially suitable to have effects on tumor diseases.

Combination

The combinations of the present invention thus can be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses, such as, for example, haematological tumours and/or metastases thereof, solid tumours, and/or metastases thereof, e.g. leukaemias, multiple myeloma thereof and myelodysplastic syndrome, malignant lymphomas, breast tumours including and bone metastases thereof, tumours of the thorax including non-small cell and small cell lung tumours and bone metastases thereof, gastrointestinal tumours, endocrine tumours, mammary and other gynaecological tumours and bone metastases thereof, urological tumours including renal, bladder and prostate tumours, skin tumours, and sarcomas, and/or metastases thereof.

One embodiment relates to the use of a combination according to any one of claims 1 to 12 for the preparation of a medicament for the treatment or prophylaxis of a cancer, particularly breast cancer, prostate cancer, multiple myeloma, hepatocyte carcinoma, lung cancer, in particular non-small cell lung carcinoma, colorectal cancer, melanoma, or pancreatic cancer.

In one embodiment the invention relates to a method of treatment or prophylaxis of a cancer, particularly breast cancer, prostate cancer, multiple myeloma, hepatocyte carcinoma, lung cancer, in particular non-small cell lung carcinoma, colorectal cancer, melanoma, or pancreatic cancer, in a subject, comprising administering to said subject a therapeutically effective amount of a combination according to any one of claims 1 to 12.

In another embodiment the invention relates to a method of treatment or prophylaxis of a cancer, particularly breast cancer, prostate cancer, multiple myeloma, hepatocyte carcinoma, lung cancer, in particular non-small cell lung carcinoma, colorectal cancer, melanoma, or pancreatic cancer, in a subject, comprising administering to said subject a therapeutically effective amount of a combination according to any one of claims 1 to 12.

In another embodiment the invention relates to a method of treatment or prophylaxis of a cancer, particularly breast cancer, prostate cancer, multiple myeloma, hepatocyte carcinoma, lung cancer, in particular non-small cell lung carcinoma, colorectal cancer, melanoma, or pancreatic cancer and/or metastases thereof in a subject, comprising administering to said subject a therapeutically effective amount of a combination according to any one of claims 1 to 12.

Preferred uses of the combinations of the invention are the treatment of multiple myeloma, lung, breast and prostate cancer, especially castration-resistant prostate cancer (CRPC).

One preferred embodiment is the use of the combinations of the invention for the treatment of prostate cancer, especially castration-resistant prostate cancer (CRPC).

One preferred embodiment is the use of the combinations of the invention for the treatment of breast cancer.

One preferred embodiment is the use of the combinations of the invention for the treatment of ovarian cancer.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Combinations of the present invention might be utilized to inhibit, block, reduce, decrease, etc., cell proliferation and/or cell division, and/or produce apoptosis.

This invention includes a method comprising administering to a mammal in need thereof, including a human, an amount of a component A and an amount of component B of this invention, or a pharmaceutically acceptable salt, isomer, polymorph, metabolite, hydrate, solvate or ester thereof; etc. which is effective to treat the disorder.

Hyper-proliferative disorders include but are not limited, e.g., psoriasis, keloids, and other hyperplasias affecting the skin, benign prostate hyperplasia (BPH), as well as malignant neoplasia. Examples of malignant neoplasia treatable with the compounds according to the present invention include solid and hematological tumors. Solid tumors can be exemplified by tumors of the breast, bladder, bone, brain, central and peripheral nervous system, colon, anum, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor. In addition, malignant neoplasias include primary tumors in said organs and corresponding secondary tumors in distant organs (“tumor metastases”). Hematological tumors can be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Combinations of the present invention might also be used for treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumor enlargement and metastasis. Moreover, the growth of new blood and lymph vessels in a tumor provides an escape route for renegade cells, encouraging metastasis and the consequence spread of the cancer. Thus, combinations of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

### Dose and Administration

Component A

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredients to be administered in the treatment of one of these conditions can vary widely according to such considerations as the particular component And dosage unit employed, the mode of administration, the period of treatment, the age and sex of the patient treated, and the nature and extent of the condition treated.

Component A being a PD-1/PD-L1 inhibitor, as described supra, can be administered to a patient at a dosage which can range from about 1 to about 2000 mg per day. Particularly, the PD-1/PD-L1 inhibitor can be administered at a dosage of 0.005 to 10 mg/kg, preferably at a dosage of 1 to 10 mg/kg by weight of patient.

Also, the agents can be administered in conventional amounts routinely used in cancer chemotherapy. Typically, the following treatments are used:

Nivolumab: Administer as an intravenous infusion over 60 minutes.
Unresectable or metastatic melanoma: 240 mg nivolumab every 2 weeks.
Unresectable or metastatic melanoma: nivolumab with ipilimumab: nivolumab 1 mg/kg, followed by ipilimumab on the same day, every 3 weeks for 4 doses, then nivolumab 240 mg every 2 weeks.
Metastatic non-small cell lung cancer: nivolumab 240 mg every 2 weeks.
Advanced renal cell carcinoma nivolumab 240 mg every 2 weeks.
Classical Hodgkin lymphoma: nivolumab 3 mg/kg every 2 weeks.

Pembrolizumab:
Melanoma: 2 mg/kg every 3 weeks.
NSCLC (=non small cell lung carcinoma): 200 mg every 3 weeks.
HNSCC (=head and neck Squamous cell carcinoma): 200 mg every 3 weeks.
cHL (=classical Hodgkin lymphoma): 200 mg every 3 weeks for adults; 2 mg/kg (up to 200 mg) every 3 weeks for pediatrics.

Atezolizumab: Administer 1200 mg as an intravenous infusion over 60 minutes every 3 weeks.

Durvalumab: 10 mg/kg as an intravenous infusion over 60 minutes every 2 weeks.

Avelumab: administer 10 mg/kg as an intravenous infusion over 60 minutes every 2 weeks. Premedicate with acetaminophen and an antihistamine for the first 4 infusions and subsequently as needed.

Of course the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Suitable dose(s), administration regime(s) and administration route(s) for component A being a PD-1/PD-L1 inhibitor include those described in the NCCN Clinical Practice Guidelines in Oncology (NCCN guidelines), in particular in the NCCN Guidelines in Oncology, Version 1.2017.

Further, suitable dose(s), administration regime(s) and administration route(s) for component A may be readily determined by standard techniques known to the skilled person.

The dose(s), administration regime(s) and administration route(s) may have to be adapted according to, inter alia, the indication, the indication stage, the patient age and/or the patient gender, among other factors. Such adaptations can be readily determined by standard techniques known to the skilled person. For both, the PD-1/PD-L1 inhibitor, particularly Compound A, and for the TTC, the administered dosage of the compound(s) may be modified depending on any superior or unexpected results which may be obtained as routinely determined with this invention.

Component B

A dosage regimen for TTC injection, for example, may be 15 kBq per kg body weight given at 6 week intervals, as a course consisting of 4 injections or more. As an example, the TTC solution may be supplied in a single-dose vial.

TTCs can be administered intravenously by qualified personnel as a slow bolus injection. An intravenous access line should be used for administration of a TTC.

Of course the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Combinations of the Present Invention

The combinations of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The combinations of component A and component B of this invention can be administered as the sole pharmaceutical agent or in combination with one or more further pharmaceutical agents C where the resulting combination of components A, B and C causes no unacceptable adverse effects. For example, the combinations of components A and B of this invention can be combined with component C, i.e. one or more further pharmaceutical agents, such as known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof.

Component C, can be one or more pharmaceutical agents such as 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 1143269, BAY 1000394, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin or combinations thereof.

Alternatively, said component C can be one or more further pharmaceutical agents selected from gemcitabine, paclitaxel, cisplatin, carboplatin, sodium butyrate, 5-FU, doxirubicin, tamoxifen, etoposide, trastumazab, gefitinib, intron A, rapamycin, 17-AAG, U0126, insulin, an insulin derivative, a PPAR ligand, a sulfonylurea drug, an a-glucosidase inhibitor, a biguanide, a PTP-1B inhibitor, a DPP-IV inhibitor, a 11-beta-HSD inhibitor, GLP-1, a GLP-1 derivative, GIP, a GIP derivative, PACAP, a PACAP derivative, secretin or a secretin derivative.

Optional anti-hyper-proliferative agents which can be added as component C to the combination of components A and B of the present invention include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use as component C with the combination of components A and B of the present invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel (when component B is not itself paclitaxel), pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use as component C with the combination of components A and B of the present invention include but are not limited to other anti-cancer agents such as epothilone and its derivatives, irinotecan, raloxifen and topotecan.

Generally, the use of cytotoxic and/or cytostatic agents as component C in combination with a combination of components A and B of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(8) provide a longer time for tumor progression, and/or
(9) yield efficacy and tolerability results at least as good as those of the agents used alone, compared to known instances where other cancer agent combinations produce antagonistic effects.

### EXPERIMENTAL SECTION

### Examples Demonstrating the Synergistic Effect of the Combinations of Components A and B of the Present Invention

Component A:

In this Experimental section, the term “compound A” refers to Atezolizumab.

Component B:

In this Experimental Section and in the Figures, the term “compound B” can be MSLN-TTC, PSMA-TTC or HER2-TTC, each of which is described above.

MSLN-TTC is BAY2287411 and is prepared according to Example 7, specifically Examples 7a and 7b of WO 2016/096843.

HER2-TTC is BAY 2331370 and is prepared according to Example 3, particularly Examples 3.1-3.4 of WO 2017/162555.

PSMA-TTC is BAY 2315497 and is prepared according to Example 9, specifically Examples 9a and 9b of WO 2016/096843. The monoclonal antibody is AB-PG1-XG1-006 as disclosed in WO 03/034903.

### Examples Demonstrating the Immunostimulatory Properties of MSLN-TTC In Vitro

The immuostimualtory effect of compound B′ on the mesothelin overexpressing human ovarian cancer cell line OVCAR-3 was demonstrated in vitro. The effects were evaluated by measuring the release of cytokines and chemokines into the supernatant after exposure of human ovarian cancer OVCAR-3 cells to MSLN-TTC. For tis purpose, cells were seeded in 12-well plates at a density of 500.000 cells/well. On day 0, compound A was incubated on cells at radioactivities of 0.5 or 5 kBq/ml. A radiolabeled isotype control at same radioactivity dose levels was included to demonstrate specificity. Further, non-radiolabeled compound B′ as well as medium only were included to measure background secretion of chemokines and cytokines. As a positive control, cGAMP (cyclic guanosine monophosphate-adenosine monophosphate, 20 μg/ml), described to activate type I interferons via the stimulator of interferon genes (STING), was included. After five days, the supernatant was harvested and analyzed using a customized mesoscale 10-plex plate, comprising detection antibodies for the following analytes:

interferon-beta (IFN-beta), interferon-gamma (IFN-gamma), interleukin-6 (IL-6), interleukin-8 (IL-8), interferon gamma-induced protein 10 (IP-10), macrophage inflammatory protein 1-alpha (MIP1a), macrophage inflammatory protein 1-beta (MIP1b), macrophage inflammatory protein 3-alpha (MIP3a) as well as tumor necrosis factor alpha (TNFa).

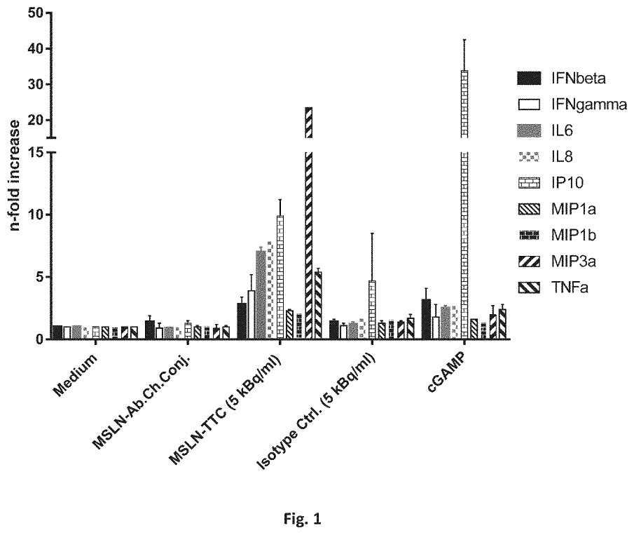

The results of this experiment are presented in FIG. 1. It was observed that several of the above described analytes showed increased levels in the supernatants compared to cells cultured in medium only. The upregulation was specific for radiolabeled compound B′ as non-radiolabeled compound B′ did not show the same effect. Slight upregulation of some analytes was also observed for a radiolabeled isotype control, but not to the same level as for compound B′, demonstrating targeting specificity. The positive control cGAMP showed strong induction of IP-10, one of the key target genes upregulated by cGAMP.

In summary the above described in vitro data demonstrate that compound B′ evokes the secretion of immunostimulatory chemokines/cytokines, some of these overlapping with STING mediated signalling. This inherent property of compound B′ may be an opportunity to combine it with other targeted immunestimulating/regulating therapies.

### Examples Demonstrating the Immunostimulatory Properties of MSLN-TTC In Vivo

In a second set of experiments, the immunostimulatory properties of compound B′ were tested in vivo in immunecompetent mice. As compound B′ (MSLN-TTC) is not cross-reactive to mouse MSLN, the murine colorectal cancer cell line MC38 was stably transfected with the human mesothelin gene, resulting in the cell line MC38-hMSLN. Cells were inoculated at a concentration of 1×10⁶ cells/mouse in 50% matrigel subcutaneously into the right flank of C57BL/6 mice (Charles River). Treatment with compound B′ was initiated on day 5 post tumor implantation (mean tumor size of 88 mm³) at a single dose level i.v. of 125, 250 and 500 kBq/kg (total antibody dose of 0.14 mg/kg). Tumor size and animal body weight were measured twice a week and the tumor volume was calculated with the formula: ((length×width²)/2). At study day 126 post tumor inoculation, tumor free survivors were re-challenged either with MC38-hMSLN cells (1×10⁶ cells/mouse in 50% matrigel) or with B16F10 cells (0.5×10⁶ cells/mouse), inoculated at the same site that the former MC38-hMSLN tumor.

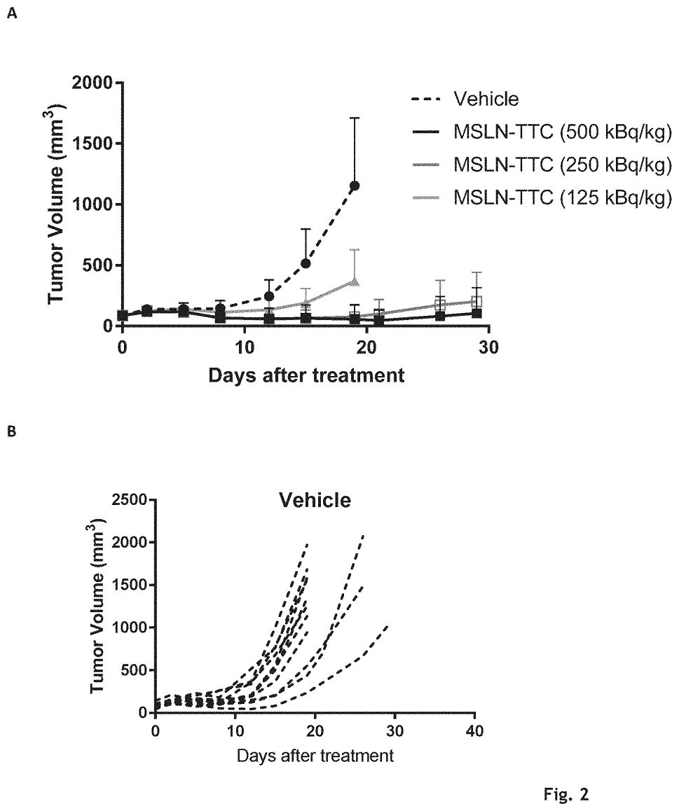

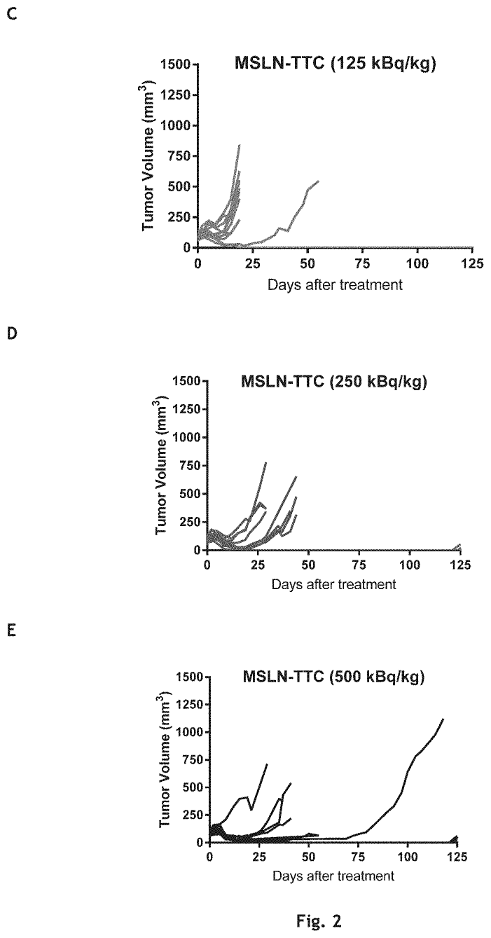

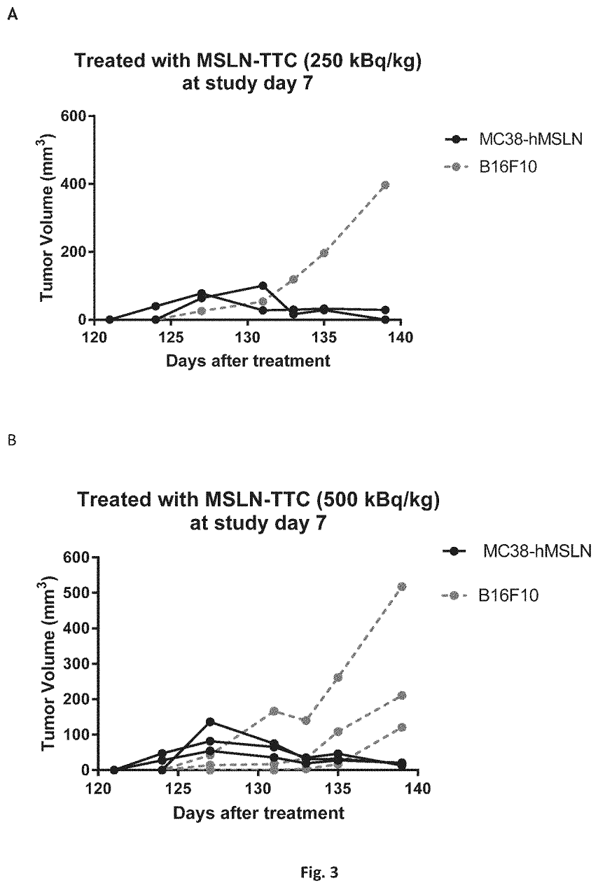

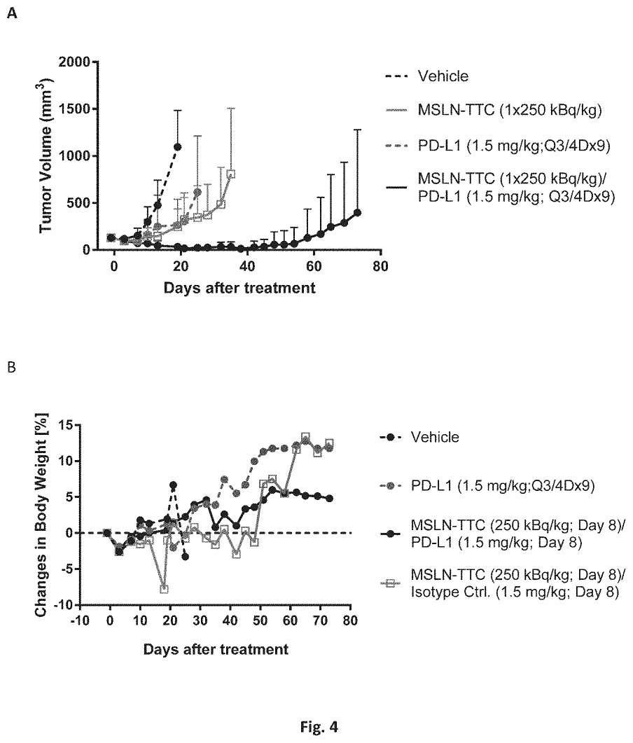

The results of this study are presented in the following FIGS. 2 and 3. Following single dose administration to MC38-hMSLN tumor-bearing mice, compound B′ induced dose dependent antitumor activity with complete tumor eradication in a total of 10 out of 33 treated animals up to study day 121 post treatment (see Table 1). Further, when tumor-free animals were re-inoculated with MC38-hMSLN cells on day 121 post treatment, no tumor growth was observed (FIG. 3). In contrast, tumors grew in animals inoculated with B16F10 cells suggesting the development of an immune memory response against MC38-hMSLN cells after treatment with compound B′.

In summary, the data demonstrate that single-dose administration of compound B′ (MSLN-TTC) to MC38-hMSLN tumor bearing immunocompetent mice resulted in dose dependent complete cures. Further, re-challenging of tumor-free survivor mice with B16F10 cells demonstrated that mice had developed and immune-memory effect against MC38-hMSLN cells, but not towards B16F10 cells. The data presented demonstrate that compound B′ is capable of evoking an immunostimulatory response in vivo and therefore may show combination potential with other immunostimulatory/regulatory therapies.

TABLE 1
Number of individual animals, which had no measurable
tumors at study day 121 after treatment.

| | Compound B′ - MSLN-TTC | | |
|---|---|---|---|
| Dose in kBq/kg | 125 | 250 | 500 |
| Number of tumor free | 1/11 | 3/11 | 6/11 |
| survivors per group | | | |

### Examples Demonstrating the Synergistic Effect of the Combinations of Compound A′ and Compound B′ of the Present Invention

In an additional experiment, the in vivo potency of compound B′ (MSLN-TTC or LRRC15-TTC) in combination with compound A′ (PD-L1, atezolizumab) was evaluated in immunocompetent mice.

For this purpose, MC38-hMSLN cells were inoculated at a concentration of 1×10⁶ cells/mouse in 50% matrigel subcutaneously into the right flank of C57BL/6 mice (Charles River). Treatment with compound B′ (MSLN-TTC) was initiated on day 8 post tumor implantation at a single dose level i.v. of 250 kBq/kg (total antibody dose of 0.14 mg/kg). In one instance, compound A′ was combined with an isotype control antibody, dosed at 1.5 mg/kg, starting on day 8 at an interval of Q3/4Dx7. In parallel, compound B′ was combined with compound A′, starting on day 8 at a dose of 1.5 mg/kg at an interval of Q3/4Dx9. As control, compound A′ was administered as monotherapy, starting on day 8 at a dose of 1.5 mg/kg at an interval of Q3/4Dx7. The tumor size was measured twice a week and the tumor volume was calculated ((length×width²)/2) and plotted as presented in FIG. 4. Efficacy was evaluated by calculating the % T/C on day 21=(mean tumor volume in treated group/mean tumor volume in vehicle control group)×100. Statistical significance was calculated on Log-transformed tumor volumes in the treated groups versus the vehicle on day 21 using One way ANOVA (Dunnett's method)

As presented in FIG. 4, monotherapy vs combination treatment of compound B′ (MSLN-TTC) with compound A′ are presented. Strong increase of in vivo efficacy was observed when compound B′ was combined with compound A′ in the MC38-hMSLN syngeneic model.

Treatment over control ratio of compound B′ (MSLN-TTC) with compound A (anti-PD-L1) in the humanized murine model MC38-hMSLN

| | Compound B′ | Compound A | combination |
|---|---|---|---|
| T/C day 21 | 0.31 (P = 0.0163 | 0.53 (P = 0.001 | 0.10 (P = 0.0001 |
| | versus vehicle) | versus vehicle) | versus vehicle) |

FIG. 1/4

Immunostimulatory effects of compound A′ (MSLN-TTC) on the human ovarian cncer cell line OVCAR-3 in vitro. Cells were incubated for five days in presence of the indicated compounds. Cell supernatants were harvested and analyzed using a customized 10-plex plate. Raw data were normalized to cells incubated in presence of medium only and data are expressed in n-fold. cGAMP was included as positive control.

FIG. 2/4

In vivo evaluation of compound B′ (MSLN-TTC) in tumor-bearing MC38-hMSLN immunocompetent mice. (A) Mean tumor volume of animals after single dose administration of compound B′ at radioactivity doses of 125, 250 and 500 kBq/kg. (B-E) Individual growth curves for each group until study day 121 after treatment.

FIG. 3/4

Re-challenge of tumor free-surviving mice (from FIG. 2 above) with either MC38-hMSLN or B16F10 cells. Individual tumor-free surviving mice treated either with 250 kBq/kg (A) or 500 kBq/kg (B) compound B′ (MSLN-TTC) at study day 7 were re-challenged with the tumor cell line B16F or MC38-hMSLN at study day 121.

FIG. 4/4

(A) Combination of compound B′ (MSLN-TTC) with compound A′ (PD-L1) in comparison to respective monotherapy treatments. Test items were administered at the respective doses as indicated into MC38-hMSLN tumor-bearing mice. (B) Changes in body weight in % after start of therapy.

### CONCLUSIONS

In the human ovarian cancer cell line OVCAR-3, compound B′ (MSLN-TTC) evokes the secretion of pro-inflammatory cytokines and chemokines when exposed for up to five days. Of note, some of the secreted factors are target molecules which get activated by the stimulator of interferon genes (STING) protein.

Using the murine colorectal cancer cell line MC38, transfected with human MSLN resulting MC38-hMSLN, it was demonstrated that compound B′ has single agent dose dependent monotherapy efficacy. Further, treatment of MC38-hMSLN tumor bearing mice seem to evoke an immunostimulatory memory response as tumor free survivors showed tumor growth protection against the MC38-hMSLN cell line when re-challenged 125 days post treatment with compound B′, but not towards a MSLN-negative cell line, B16F10.

When compound B′ (MSLN-TTC), administered as a single dose, was combined with compound A′ (PD-L1), administered simultaneously at a dosing schedule of QD3/4x9, strong increase in in vivo efficacy was observed in MC38-hMSLN tumor bearing mice.

In summary, our data indicate that compound B′ has a immunostimulatory effect in monotherapy. Further, compound B′ shows additive/synergistic effect in vivo when combined with compound A′. Further clinical evaluation of this promising combination therapy for the treatment of cancer is warranted.

1. A combination, comprising a component A, wherein component A is a PD-1/PD-L1 inhibitor, and component B, wherein component B is a targeted thorium conjugate.

2. The combination according to claim 1, wherein the PD-1/PD-L1 inhibitor is selected from the group consisting of nivolumab, pembrolizumab, PDR-001, JS001, STI-A1110, atezolizumab, durvalumab, avelumab, BMS-936559 and LY3300054.

3. The combination according to claim 1, wherein the PD-1/PD-L1 inhibitor is selected from the group consisting of nivolumab, pembrolizumab, atezolizumab, durvalumab and avelumab.

4. The combination according to claim 1, wherein the targeted thorium conjugate is selected from the group consisting of MSLN-TTC, PSMA-TTC and HER2-TTC.

5. The combination according to claim 1, wherein the PD-1/PD-L1 inhibitor is atezolizumab and the targeted thorium conjugate is MSLN-TTC.

6. (canceled)

7. (canceled)

8. A method of treatment or prophylaxis of a cancer in a subject, comprising administering to said subject a therapeutically effective amount of a combination according to claim 1, wherein the cancer is breast cancer, prostate cancer, multiple myeloma, hepatocyte carcinoma, lung cancer, colorectal cancer, melanoma, or pancreatic cancer and/or metastases thereof.

9. A kit comprising a combination according to claim 1,
wherein both or either of the PD-1/PD-L1 inhibitor and the targeted thorium conjugate are in the form of a pharmaceutical formulation which is ready for use to be administered simultaneously, concurrently, separately or sequentially.

10. A composition, comprising a combination according to claim 1, and pharmaceutically acceptable ingredients.

11. The method of claim 8, wherein the cancer is multiple myeloma, lung, breast and prostate cancer, and/or metastases thereof.

12. The method of claim 11, wherein the cancer is prostate cancer is castration-resistant prostate cancer (CRPC) or the lung cancer is non-small cell lung carcinoma.

13. The method of claim 8, wherein the cancer is hepatocyte carcinoma, lung cancer, colorectal cancer, melanoma, pancreatic cancer, prostate cancer, breast cancer and/or metastases thereof.

14. The kit according to claim 9, wherein the combination comprises one or more further pharmaceutical agents C.
